(19) United States
(12) Patent Application Publication
(10) Pub. No.: US 2026/0315044 A1
(43) Pub. Date: Oct. 15, 2026

(54) PUNCTUM-EMBEDDED DEVICE AND SYSTEM FOR OCULAR BIOMARKER SENSING, DIAGNOSTIC, DRUG DELIVERY, AND THERAPEUTICS, INCORPORATING THE ACCURATE-UNDER-IMPERFECT-CONDITIONS (AcUIC) SENSING AND ASSAY METHODOLOGY

(71) Applicant: Stephen Y. Chou, Princeton, NJ (US)

(72) Inventor: Stephen Y. Chou, Princeton, NJ (US)

(21) Appl. No.: 19/643,517

(22) Filed: Apr. 9, 2026

Related U.S. Application Data
(60) Provisional application No. 63/785,930, filed on Apr. 9, 2025.
(60) Provisional application No. 63/785,951, filed on Apr. 9, 2025.
(60) Provisional application No. 63/785,970, filed on Apr. 9, 2025.

(51) Int. Cl. A61B 10/00; A61B 5/00; A61B 5/1473; A61F 9/00

(52) U.S. Cl. CPC A61B 10/0045; A61B 5/0004; A61B 5/1473; A61B 5/4839; A61B 5/6821; A61B 5/7221; A61B 5/7267; A61F 9/0017; A61B 2010/0067; A61B 2562/0285

(57) ABSTRACT

A punctum-embedded ocular care platform, i-NanoOcuCare, integrates continuous tear biomarker sensing, closed-loop drug delivery, and AI-driven therapeutics within millimeter-scale punctal plugs. A sensing plug (i-Sense) comprises a nano sensor array, microfluidic pump, active valve, 3D-packaged integrated circuit for wireless communication and energy harvesting, and machine learning software implementing the Accurate Under Imperfect Conditions (AcUIC) paradigm with Error Risk Analysis (ERA) for clinically reliable measurement accuracy. A drug delivery plug (i-Release) delivers controlled therapeutic doses under instructions from a therapeutic algorithm (i-TheraX). A deployable flexible intracanalicular antenna achieves up to 100-fold greater wireless RF energy harvesting efficiency than antennas confined to the plug body. The system communicates wirelessly with a pocket-sized station, cloud platform, and wearable devices, enabling fully closed-loop AI-driven ocular monitoring and treatment. AcUIC methodology is independently described and reserved as standalone continuation claims.

### CROSS-REFERENCE TO RELATED APPLICATIONS

The present application claims the benefit of U.S. Provisional Patent Application No. 63/785,930, filed Apr. 9, 2025, entitled “Detection and Monitoring of Ocular Protein in Tear”; U.S. Provisional Patent Application No. 63/785,951, filed Apr. 9, 2025, entitled “Punctum-Embedded Device and Its System for Ocular Biomarker Sensing, Diagnostic, Drug-Delivery, and Therapeutics”; and U.S. Provisional Patent Application No. 63/785,970, filed Apr. 9, 2025, entitled “Accurate Periocular Assay Under Imperfect Conditions,” the entire disclosures of which are incorporated herein by reference in their entirety.

### TECHNICAL FIELD

The present disclosure relates generally to medical devices and systems for ophthalmic care. More particularly, the invention relates to a miniaturized, punctum-embedded platform—termed i-NanoOcuCare—that integrates continuous tear biomarker sensing, closed-loop drug delivery, AI-driven diagnostic algorithms, and wireless communication, all housed within a millimeter-scale plug configured to be implanted in the lacrimal punctum of the eye.

The disclosure further relates to the Accurate Under Imperfect Conditions (AcUIC) sensing methodology—a system architecture, method, and algorithm framework that enables clinically accurate periocular biomarker assay results in the inherently imperfect in vivo punctal environment—and to the Error Risk Analysis (ERA) algorithm that is a central component of AcUIC. The AcUIC methodology is described and claimed both as an integral subsystem of i-NanoOcuCare and, in the Continuation-Reserved Claims of Section XVI, as a standalone sensing platform.

### BACKGROUND

Ocular diseases such as dry eye disease (DED), glaucoma, diabetic retinopathy, and infectious keratitis affect hundreds of millions of people worldwide. Effective management of these conditions requires, ideally, continuous monitoring of relevant biomarkers in the tear film combined with precisely timed and dosed therapeutic interventions. Tear fluid is a rich source of biomarkers—including osmolarity, glucose, lactate, urea, cytokines, electrolytes, and various proteins—whose concentrations reflect local and systemic disease states. However, clinically actionable, real-time tear biomarker monitoring has remained elusive.

Existing approaches to ocular drug delivery include topical eye drops, ocular inserts, and intravitreal injections, each of which suffers from significant limitations. Eye drops have low bioavailability and require frequent patient-administered dosing. Passive ocular inserts cannot adapt delivery in response to changing physiological conditions. Intravitreal injections are invasive and carry risks of infection and retinal damage. None of these approaches is capable of integrating continuous biomarker monitoring with real-time, feedback-driven drug delivery.

The lacrimal punctum—the small opening at the medial canthus of each eyelid through which tears drain—offers a uniquely attractive site for a miniaturized, long-dwelling medical device. Punctal plugs have been used clinically for decades to treat DED by blocking tear drainage, demonstrating excellent biocompatibility and patient tolerance. The punctum is in direct, continuous contact with the tear film, providing access to tear biomarkers in their native, undiluted state. Furthermore, the nasolacrimal canal connected to the punctum provides a conduit through which an elongated antenna could be deployed to substantially improve wireless energy harvesting and data transmission performance.

Despite this favorable anatomy, prior art punctal plug devices have been limited to passive tear occlusion or simple drug elution. No prior art device combines, within a single punctum-embedded platform: (i) active, multi-biomarker sensing with closed-loop calibration; (ii) controlled, feedback-driven drug delivery; (iii) an onboard artificial intelligence engine; (iv) integrated wireless power harvesting and data communication; and (v) a deployable intracanalicular antenna for enhanced wireless performance.

A further and critical deficiency of all prior art periocular sensor designs is the assumption of near-ideal test conditions. In practice, sensors operating continuously in the lacrimal environment face: varying tear volumes dependent on blink rate and lacrimal secretion dynamics; temperature fluctuations from ambient conditions and local tissue perfusion; evaporative concentration of analytes; protein fouling of electrode surfaces; reagent aging and degradation; and variable ionic background composition. Standard calibration approaches are entirely insufficient to maintain acceptable measurement accuracy under these continuously varying conditions.

The present invention addresses both deficiencies through: (1) the i-NanoOcuCare platform, which provides the first fully integrated punctum-embedded sensing, drug delivery, and therapeutic system; and (2) the AcUIC paradigm and ERA algorithm, which provide the first systematic, quantitative framework for achieving clinically accurate biomarker measurements in the inherently imperfect punctal environment.

The magnitude of this unmet need is confirmed by two independent third-party sources. First, a 2025 state-of-the-art review of smart ophthalmic biomaterials (Li et al., Biomaterials, 2025) explicitly characterized the current state of the art as “not yet fully autonomous or AI-driven” and as lacking “closed-loop, autonomous control”—precisely the capabilities provided by the present invention. Second, the U.S. Advanced Research Projects Agency for Health (ARPA-H) launched its Ocular Laboratory for Analysis of Biomarkers (OCULAB) program in 2024, explicitly seeking to develop a tear-based biomarker measurement system insertable into the tear duct for continuous health monitoring and ultra-precise closed-loop medication dosing—a technical goal that corresponds directly to the i-NanoOcuCare platform claimed herein. The fact that a U.S. federal research agency identified this as a research-grade challenge requiring government-funded innovation, contemporaneously with the present invention, constitutes strong objective evidence that the present invention was non-obvious to persons of ordinary skill at the time of conception.

### BRIEF SUMMARY

In a first aspect, the present invention provides a punctum-embedded ocular sensing device comprising: a punctal plug configured for implantation within the lacrimal punctum; at least one sensing channel allowing tear fluid flow through the plug; a nano sensor array configured to detect one or more biomarkers in the tear fluid; a microfluidic pump and active fluidic valve controlling tear fluid flow through the sensing unit; an integrated circuit (IC) chip providing wireless communication, energy harvesting, and electronic signal control; and software comprising machine learning algorithms implementing the AcUIC paradigm—including ERA—to produce biomarker measurements that are accurate under imperfect tear sampling conditions.

In a second aspect, the invention provides a closed-loop ocular care system comprising: a sensing plug (i-Sense) for continuous tear biomarker detection; a drug delivery plug (i-Release) for controlled periocular drug release; a therapeutic algorithm subsystem (i-TheraX) generating dosing instructions from biomarker data; and a wireless communication station for bidirectional communication with both plugs, cloud infrastructure, and wearable devices; wherein all components operate in concert in a fully closed-loop, AI-driven ocular monitoring and treatment system.

In a third aspect, the invention provides a method for closed-loop ocular monitoring and treatment comprising: implanting a sensing plug within a lacrimal punctum; continuously drawing tear fluid through the sensing channel; detecting biomarker signals using the nano sensor array; processing signals using the AcUIC ML algorithm to produce imperfection-compensated biomarker measurements; applying ERA to verify measurement reliability; generating and transmitting dosing instructions via i-TheraX; and delivering a therapeutic agent via the drug delivery plug.

In a fourth aspect, the invention provides a deployable intracanalicular antenna system comprising an elongated, flexible antenna element configured to extend from a punctal plug into the lacrimal canaliculus, wherein wireless energy harvesting efficiency increases quadratically with antenna length, enabling up to approximately 100-fold greater RF power harvesting compared to an antenna confined to the punctal plug body.

In a fifth aspect, the invention provides, as a standalone invention, the AcUIC periocular biomarker assay system comprising: at least one primary sensing element; a microfluidic control subsystem for sample preparation and renewal; a plurality of condition-monitoring elements; an ERA module computing a risk factor for assay accuracy; and a decision module that either delivers a corrected assay result or discards the result and initiates a new assay cycle based on comparison of the risk factor to a predefined threshold.

### BRIEF DESCRIPTION OF THE DRAWINGS

The accompanying drawings, which are incorporated in and constitute a part of this specification, illustrate preferred embodiments of the invention.

FIG. 1A is a schematic overview of the i-NanoOcuCare system of the present disclosure, showing the millimeter-scale i-NanoOcuCare punctal plug implanted in the lacrimal punctum of the eye, the three integrated subsystems housed within the plug—i-Sense (106), i-Release (108), and SOC (110)—their internal connections, the wireless link from SOC to the i-TheraX pocket-sized wireless station (114), and the external network comprising a cloud computing platform (116), a smartwatch (118), and a medical professional (120), including a direct wireless link between the smartwatch and the wireless station. An enlarged detail view of the lacrimal punctum shows the plug inserted with its cap approximately flush with, or slightly proud of, the punctum surface, and the plug body extending into the canaliculus.

FIG. 1B is a schematic overview of the functions of the i-NanoOcuCare system of the present disclosure, showing the millimeter-scale punctal plug (202) implanted in the lacrimal punctum of the eye and the four integrated functions of the platform: continuous tear biomarker sensing by i-Sense (204), closed-loop drug delivery by i-Release (206), AI-driven diagnostic algorithms by i-TheraX (208), and wireless communication and power (210), interconnected by internal data and command links and by external wireless connections to form a closed-loop, AI-controlled ophthalmic care system.

FIG. 2 is a schematic of the i-NanoOcuCare punctal plug structure, comprising a longitudinal cross-section (upper portion) showing the posterior retention disk (308), the central tubular body (306) defining a sensing channel (312) containing the sensing unit (302) and a bypass channel (314) containing an optional capillary pump (316), and the front insertion cone (310), with tear fluid flow paths and illustrative dimensions; and a transverse cross-section A-A (lower portion) showing the two-channel configuration of the tubular body in end-on view.

FIGS. 3A-3B are a schematic of the multi-channel sensor array of the i-Sense subsystem, comprising a perspective view (upper portion) showing N parallel channels (402) each containing an inlet valve (406), a reference calibration element (412), an analyte-specific sensing element (410), and an outlet valve (408), mounted on a common manifold (414), with tear fluid entering from above (416) and exiting below (418) through each channel; and a single-channel detail view (lower portion) showing the internal arrangement of the inlet valve, reference element, two sensing elements (410a, 410b), and outlet valve in series along the tear fluid flow path.

FIGS. 4A-4B are schematics of the extendable antenna of the i-NanoOcuCare system, showing the punctal plug (502) positioned within the lacrimal punctum (518) with the retention disk (504) approximately flush with the eyelid surface, the plug body (506) and sensing chip (508) in the vertical canaliculus (514), and the flexible extendable antenna (510) extending from the antenna connection point (520) through the canaliculus curve and along the horizontal canaliculus (516), terminating in a guiding ball (512); with illustrative dimension annotations for plug length and maximum antenna extension, an energy harvesting note, and two insets showing the fixed and insertable antenna configurations.

FIGS. 5A-5C is a schematic of the i-Release closed-loop drug delivery plug of the present disclosure, comprising a longitudinal cross-section (upper portion) showing the retention disk (602) with SoC chip (618) and wireless communication element (628), the drug channel (604) containing the solid drug reservoir lining (606), mixing chamber (608), acoustic pump (610), electrowetting inlet valve (612), and electrowetting outlet valve (614), and the bypass channel (616) with passive capillary pump (620), with tear fluid inlet (624) and drug solution outlet (626) flow paths; and two drug chamber configuration insets (lower portion) showing the solid drug formulation (a) and liquid drug formulation (b) configurations.

### DETAILED DESCRIPTION OF THE PREFERRED EMBODIMENTS

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. The terminology used herein is for the purpose of describing particular embodiments only and is not intended to be limiting of the invention.

### Overview of the i-NanoOcuCare Platform

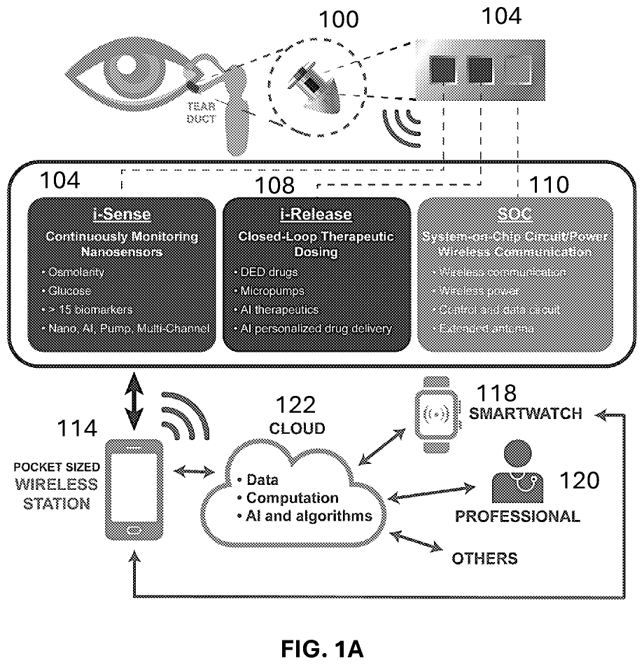

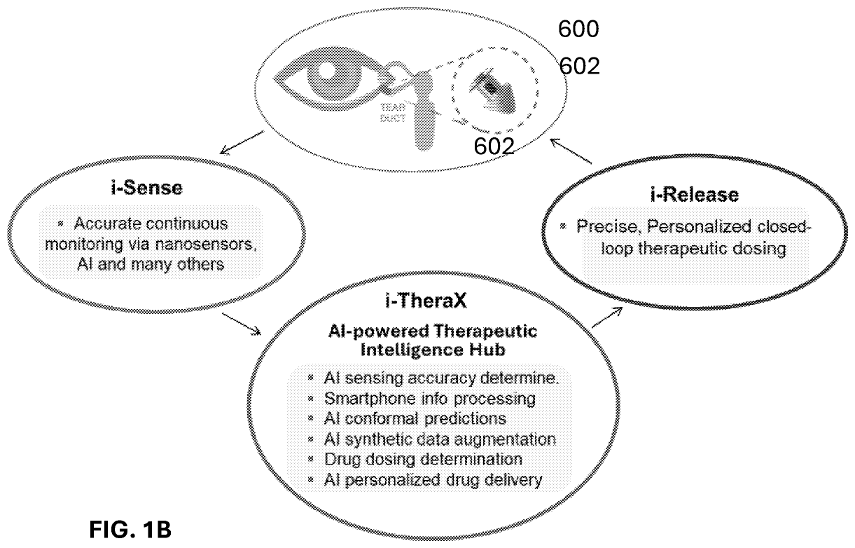

The i-NanoOcuCare platform (also referred to herein as “the device,” “the system,” or “NanoOcuCare”) is a punctum-embedded, closed-loop ocular care system. As illustrated generally in FIGS. 1A-1B, i-NanoOcuCare performs noninvasive, continuous, and closed-loop ocular monitoring, diagnosis, and drug delivery—providing comprehensive ocular care—through a millimeter-sized plug embedded within the lacrimal punctum. The platform is wirelessly assisted by a pocket-sized wireless station further connected to cloud computing infrastructure, smartwatches, smartphones, and other wireless devices.

i-NanoOcuCare comprises three principal inter-related functional subsystems: (1) i-Sense, an intelligent continuous tear biomarker sensing subsystem; (2) i-Release, an intelligent closed-loop drug delivery subsystem; and (3) i-TheraX, a therapeutic algorithm subsystem. These subsystems, together with wireless communication, AI and ML engines, cloud connectivity, and smartwatch/phone integration, constitute the complete i-NanoOcuCare platform.

Overview. i-Sense—a key component of i-NanoOcuCare. i-Sense performs intelligent, noninvasive, automatic, accurate, and continuous monitoring of tear biomarkers. i-Sense is configured to monitor more than fifteen biomarkers (Table 1) across four categories: osmolarity, small molecules, electrolytes, and proteins. Detection is achieved using four assay types: osmometry, enzyme-based sensors, ion-selective electrodes, and immunosensors. Temperature sensors and additional categorization sensors are incorporated in the channel. Nanotechnology is employed in the sensor design; for electrochemical devices, reduced dimensions yield higher sensitivity and lower power consumption.

i-Sense Plug Architecture and Innovations

The i-Sense punctal plug design in the disclosure (FIGS. 1A-5C) will fit inside the eye's punctum and has dimensions similar to traditional plugs—about 3 mm in length, with a 1 mm diameter tube and 2 mm front and rear disks. Its architecture has the following innovative features and associated advantages:

Dual Microfluidic Channels: The plug contains two tear-flow channels—a sensing channel and an optional bypass channel. The bypass allows tears to flow from the eye to the nasolacrimal duct even when the sensing channel is isolated, which improves measurement accuracy and device control. The dual channel design also allows the sensing channel to be closed most of the time to avoid the tears wash and foul the sensor, greatly enhancing the sensor lifespan.

Multi-Sub-Channel Sensing: The sensing channel includes multiple sub-channels, each equipped with its own intelligent sensing unit, pump, and electrowetting valve. This setup allows independent operation, enables start-stop tear flow during sensing, and offers redundancy—if one channel fails, another can be activated. Tear isolation also helps extend sensor life.

Intelligent Sensor Array (AcUIC+AI): The sensing chip contains an array of AcUIC intelligent sensors powered by AI to ensure accurate detection under imperfect conditions. These sensors are embedded within the sensing channel.

Acoustic Pump: Integrated into the sensing unit, the acoustic pump enables low-power, low-voltage operation while generating controlled turbulence for effective mixing and antifouling.

Passive Tear Accelerator in Bypass: A capillary-driven passive pump may be placed in the bypass channel to accelerate tear flow, compensating for flow reduction caused by sensing activity.

SoC on End Cap: A System-on-Chip (SoC) is mounted on the top surface of the end disk, providing power management, analog/digital signal processing, remote communication, and operational control. It fits within the plug's 2 mm diameter cap.

Tear Sink Inlet: Beneath the SoC, a tear sink (cross-or line-shaped) channels incoming tears into both the sensing and bypass channels.

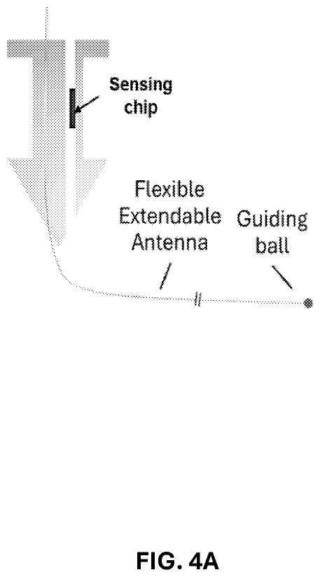

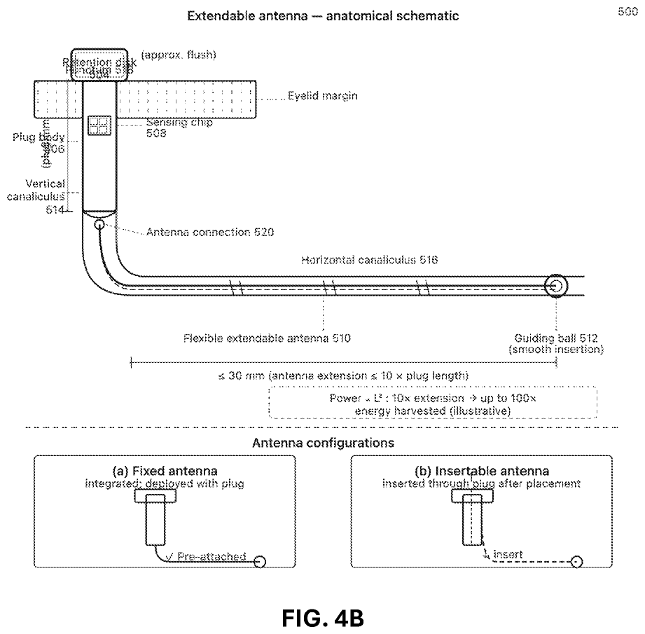

Extendable Antenna: An external, extendable antenna enhances wireless communication and energy harvesting. By extending the antenna up to 10× the plug's internal length (approx. 3 mm), the system can increase harvested energy by up to 100×, as power scales with the square of antenna length. The antenna features a guiding ball at its end to facilitate smooth insertion into the punctum. The antenna can either be fixed to the plug or inserted through the plug after the plug is positioned within the punctum (FIGS. 4A-4B).

3D Chip Integration: The chip is positioned on the plug's end cap to allow a larger footprint (~1.4×1.4 mm). 3D packaging allows large 2D chips to be packaged within the plug. Further development will explore increasing the end cap diameter to further relax chip size constraints and enhance capabilities.

FIG. 1A is a schematic overview of the i-NanoOcuCare system of the present disclosure. The i-NanoOcuCare platform is a millimeter-scale punctal plug device, designated element 100, configured for implantation in the lacrimal punctum of the eye of a subject. As shown in the enlarged detail view (element 200) in the upper right portion of FIG. 1A, the punctal plug comprises an external cap that sits flush with or slightly proud of the punctum opening at the eyelid margin, and a tapered body that is inserted into and retained within the lacrimal canaliculus. The plug body houses three distinct functional subsystems arrayed along its length within the canaliculus: i-Sense (element 106), i-Release (element 108), and SOC (element 110), described in detail below. The plug is wirelessly powered, as indicated in FIG. 1A.

Element 104 depicts the integrated device as a whole, comprising the three subsystems. Element 106 depicts the i-Sense subsystem, which continuously measures concentrations of non-protein biomarkers in the tear fluid flowing through and around the punctal plug, using nano-enabled, AI-driven, multi-channel nanosensors with micropump-assisted sample control, capable of monitoring more than fifteen distinct biomarkers simultaneously. Element 108 depicts the i-Release subsystem, which delivers therapeutic agents to the ocular surface under AI-personalized, micropump-driven closed-loop dosing control, receiving dosing commands from element 110. Element 110 depicts the SOC (System-on-Chip) subsystem, which executes AI-driven diagnostic algorithms, manages wireless communication and wireless power harvesting, and provides overall control and data processing for the device. As shown in FIG. 1A, i-Sense (106) and i-Release (108) are each connected bidirectionally to SOC (110) by internal data and command links within the plug body, whereby i-Sense transmits continuously measured biomarker concentration data to SOC, and SOC transmits therapeutic dosing commands to i-Release.

Element 112 depicts a wireless link by which SOC (110) communicates bidirectionally with element 114, i-TheraX, a pocket-sized wireless station such as a smartphone or dedicated handheld device. The wireless link 112 exits SOC downward through the center of the device and routes externally to i-TheraX. i-TheraX receives sensing data from the device, processes it locally or via the cloud computing platform (element 116) using AI-powered algorithms, determines biomarker concentrations, generates therapeutic dosing instructions, and transmits those instructions back to the device via the same wireless link.

Element 116 depicts a cloud computing platform that provides data storage, AI algorithm execution, and computational resources, and is connected bidirectionally to i-TheraX (114). Element 118 depicts a smartwatch or other wearable device that is connected bidirectionally both to the cloud (116) and, via direct wireless link element 122, directly to i-TheraX (114), enabling the smartwatch to transmit physiological and contextual data—such as heart rate, activity level, and sleep state—directly to i-TheraX without requiring routing through the cloud. Element 120 depicts a medical professional who accesses the system through the cloud (116), enabling remote monitoring of the subject's biomarker profile and therapeutic response, and remote adjustment of therapeutic parameters.

Together, elements 100 through 122 form a fully integrated, wirelessly powered, AI-controlled closed-loop ophthalmic care platform in which continuous tear biomarker sensing by i-Sense, AI-driven diagnostic processing by SOC and i-TheraX, and precise therapeutic drug delivery by i-Release operate as a unified system for the continuous, autonomous management of ocular surface disease.

FIG. 1B is a schematic overview of the four integrated functions of the i-NanoOcuCare platform of the present disclosure. Element 200 denotes the i-NanoOcuCare platform as a whole. Element 202 depicts the millimeter-scale i-NanoOcuCare punctal plug implanted in the lacrimal punctum of the eye of a subject, in fluidic contact with the tear fluid of the ocular surface. As described in connection with FIG. 1A, the plug is configured for implantation with its cap positioned approximately flush with, or slightly proud of, the punctum surface, and its tapered body retained within the lacrimal canaliculus. The plug houses, within its body, all four functional elements of the platform described below.

Element 204 depicts the i-Sense function of the platform, which provides accurate, continuous monitoring of non-protein biomarkers in the tear fluid of the subject. The i-Sense function employs nano-enabled, AI-driven, multi-channel nanosensors capable of simultaneously measuring concentrations of more than fifteen distinct non-protein biomarkers, including electrolytes, metabolites, and other analytes as described herein. Sample handling and delivery within the plug are managed by an integrated micropump, which ensures that fresh tear fluid is continuously presented to the sensing elements, maintaining measurement accuracy over extended periods of continuous operation. The sensing data generated by i-Sense are transmitted in real time to the i-TheraX function (element 208) for AI-driven processing and analysis.

Element 206 depicts the i-Release function of the platform, which provides precise, personalized, closed-loop drug delivery to the ocular surface. The i-Release function comprises a drug reservoir integrated within the plug body and a micropump-driven drug release mechanism that releases therapeutic agent in response to dosing instructions received from the i-TheraX function (element 208). The dosing instructions are generated by AI-powered algorithms that determine the appropriate therapeutic dose and timing based on the biomarker concentrations measured by i-Sense (element 204) and processed by i-TheraX. In certain embodiments, the therapeutic agent is an anti-inflammatory drug for the treatment of dry eye disease (DED) or other ocular surface conditions. The bidirectional closed-loop link between i-Sense (204) and i-Release (206), shown as element 214 in FIG. 1B, represents the complete closed-loop sensing-to-therapy cycle of the platform, wherein continuously updated biomarker measurements continuously update the therapeutic dosing in real time.

Element 208 depicts the i-TheraX function of the platform, which provides AI-driven diagnostic and therapeutic algorithm execution. The i-TheraX function receives sensing data from i-Sense (204) and applies AI-powered algorithms to determine biomarker concentrations—including the prediction of targeted protein biomarker concentrations from non-protein biomarker measurements as described herein—to assess disease state, and to generate precise, personalized therapeutic dosing instructions for i-Release (206). The i-TheraX function further incorporates conformal AI prediction algorithms, synthetic data augmentation for model robustness, and AI-driven personalized drug delivery optimization. In certain embodiments, the i-TheraX function is executed on an onboard system-on-chip (SOC) processor within the plug, on a pocket-sized external wireless station, on a cloud computing platform, or on any combination thereof, as described in connection with FIG. 1A. The dosing instructions generated by i-TheraX are transmitted to i-Release via element 212, the dosing instruction link shown in FIG. 1B.

Element 210 depicts the wireless communication and power function of the platform, which provides external connectivity and energy supply to the implanted device. The wireless communication function enables bidirectional data exchange between the implanted plug and external devices including a pocket-sized wireless station, a cloud computing platform, a smartwatch, and a medical professional, as described in detail in connection with FIG. 1A. The wireless power function delivers energy to the implanted device without requiring a battery or wired connection, enabling indefinite continuous operation of the sensing and drug delivery functions within the plug. In certain embodiments, wireless power delivery employs near-field communication (NFC), radio-frequency energy harvesting, or inductive coupling through the tissues of the periocular region.

Together, the four functions depicted in FIG. 1B—continuous tear biomarker sensing (204), closed-loop drug delivery (206), AI-driven diagnostic algorithms (208), and wireless communication and power (210)—are integrated within the millimeter-scale i-NanoOcuCare punctal plug (202) to form a unified, autonomous, continuously operating closed-loop ophthalmic care platform. The platform requires no manual intervention by the subject or clinician during normal operation, and all sensing, computation, drug delivery, and communication functions are powered and controlled wirelessly.

### i-Sense: Continuous Tear Biomarker Sensing Subsystem

### A. Structural Overview

i-Sense is an intelligent, accurate, and continuous tear biomarker monitoring device shaped as a plug configured for implantation within the lacrimal punctum of the eye. i-Sense comprises both hardware and software components. The hardware performs sensing of biomarkers in tear fluid, while the software controls and analyzes the sensing process to ensure accuracy and clinical trustworthiness.

### B. Punctal Plug Structure

The i-Sense hardware comprises a punctal plug configured to be inserted and retained within the lacrimal punctum. In a preferred embodiment, the punctal plug is approximately 3 mm in length, has a central tubular body approximately 1 mm in diameter, and is provided with a front insertion cone and a posterior retention disk, each approximately 2 mm in diameter. These dimensions are illustrative; all dimensions may be varied to accommodate individual anatomical variation and clinical requirements.

The punctal plug defines at least two channels extending longitudinally therethrough:
(a) Sensing Channel (First Channel): The first channel is a sensing channel through which tear fluid is directed for biomarker sensing. The sensing unit is disposed within the sensing channel.
(b) Bypass Channel (second Channel, Optional): the Second Channel Is a Bypass

channel that allows tear fluid to flow from the conjunctival sac to the nasolacrimal duct without passing through the sensing channel. This preserves natural tear drainage, reducing potential discomfort and maintaining ocular surface homeostasis. A passive microfluidic flow accelerator (a power-free capillary pump) may optionally be disposed within the bypass channel.

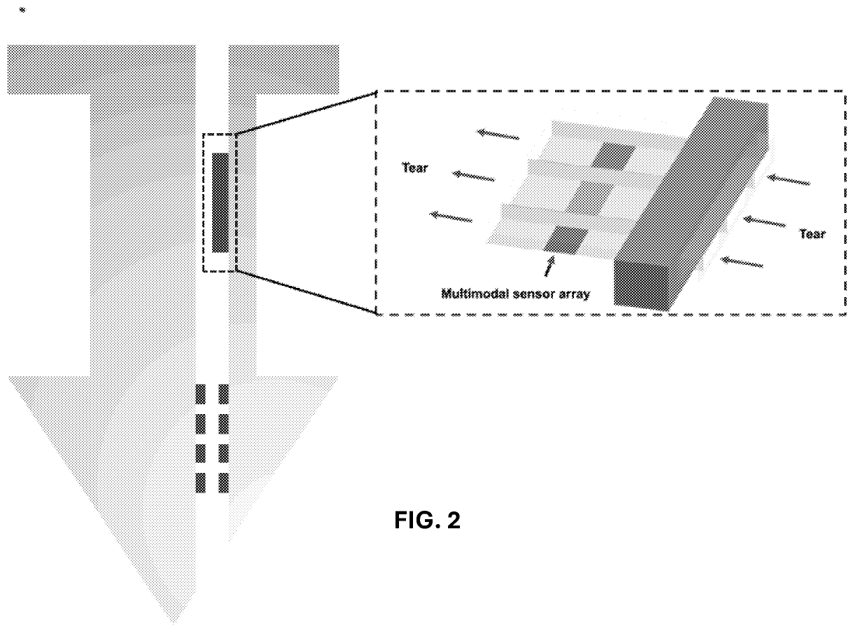

FIG. 2 illustrates the structure of the i-NanoOcuCare punctal plug of the present disclosure. The upper portion of FIG. 2 shows a longitudinal cross-section of the plug taken along the central axis, with the external (posterior) end at the left and the internal (anterior) end at the right. The lower portion of FIG. 2 shows a transverse cross-section taken at section A-A through the midpoint of the tubular body, illustrating the two-channel configuration in end-on view.

Element 308 depicts the posterior retention disk, a disk-shaped feature having a diameter of approximately 2 mm that is positioned approximately flush with, or slightly proud of, the eyelid surface at the lacrimal punctum, thereby preventing the plug from migrating further into the canaliculus after insertion. The sensing array (element 302) is disposed on the external face of the retention disk, presenting the sensing elements directly to the tear film of the conjunctival sac. Element 306 depicts the central tubular body, which has a diameter of approximately 1 mm and is sized to be retained within the lacrimal canaliculus between the retention disk and the insertion cone. Element 310 depicts the front insertion cone, a tapered feature having a maximum diameter of approximately 2 mm at its base, which facilitates atraumatic insertion of the plug into the lacrimal punctum and provides a secondary retention function by engaging the punctum walls. The total length of the assembled plug is approximately 3 mm. All dimensions are illustrative; each dimension may be varied to accommodate individual anatomical variation among subjects and to satisfy clinical requirements.

As shown in FIG. 2, the tubular body (306) defines two channels extending longitudinally therethrough, separated by a septum. Element 312 depicts the sensing channel, which is the first channel, through which a portion of the tear fluid is directed for biomarker sensing. The sensing unit (element 302) is disposed within the sensing channel, such that tear fluid passing through the sensing channel is brought into contact with the sensing elements for continuous non-protein biomarker measurement. Element 314 depicts the bypass channel, which is the second channel, through which tear fluid flows from the conjunctival sac to the nasolacrimal duct without passing through the sensing channel. The bypass channel thereby preserves the natural tear drainage pathway, reducing potential patient discomfort and maintaining ocular surface homeostasis during device operation. Element 316 depicts an optional passive microfluidic flow accelerator, a capillary pump requiring no external power source, which may be disposed within the bypass channel to enhance tear fluid throughput and improve sample renewal at the sensing unit. As shown by the flow arrows in FIG. 2, tear fluid entering the plug from the external (conjunctival sac) side splits between the sensing channel (312) and the bypass channel (314), and both streams exit toward the nasolacrimal duct at the internal (anterior) end. The transverse cross-section A-A shown in the lower portion of FIG. 2 illustrates the relative positions of the sensing channel (312) and bypass channel (314) within the plug body cross-section, with the sensing unit (302) occupying the sensing channel and the optional capillary pump (316) disposed within the bypass channel.

### C. Sensing Unit

The sensing unit is disposed within the sensing channel and comprises: (a) a nano sensor array comprising a plurality of individual sensors; (b) intelligent nanodevices for signal calibration, condition monitoring, error detection and correction, and trustworthiness verification; (c) a microfluidic pump; and (d) an active fluidic valve.

The nano sensor array is configured to detect a plurality of biomarkers simultaneously or in multiplexed fashion. In preferred embodiments, the sensing unit is capable of detecting at least 15 distinct biomarkers in four classes: (i) osmolarity, measured by an electrical conductivity osmometer; (ii) small molecule biomarkers including glucose, lactate, and urea, measured by enzyme-based electrochemical sensors (EAEC); (iii) electrolytes, measured by ion-selective electrode (ISE) sensors; and (iv) protein biomarkers, measured by continuous immunoassay sensors utilizing antibody detection agents and transistor-based binding detection, enhanced by AC electric field-driven antibody regeneration to enable continuous rather than single-point immunoassay measurements.

The sensing unit contains a plurality of identical parallel sensing sub-channels. In one operational mode, only a single sub-channel is opened for measurement at any given time while the remaining sub-channels are kept closed, thereby preventing continuous flow through all sub-channels and reducing reagent degradation and sensor fouling.

The microfluidic pump and active fluidic valve work in concert to control tear fluid flow into, through, and out of the sensing unit: blocking flow when no measurement is required; stabilizing volume within the sensing unit during measurement; and pumping out spent tear fluid after measurement to clear the sensing unit.

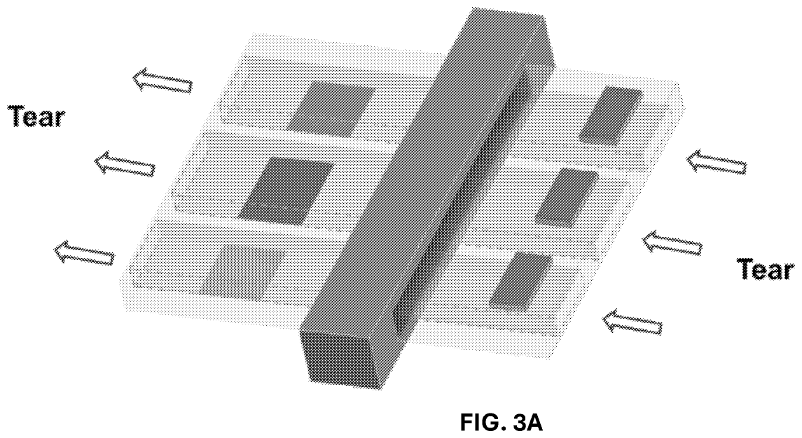

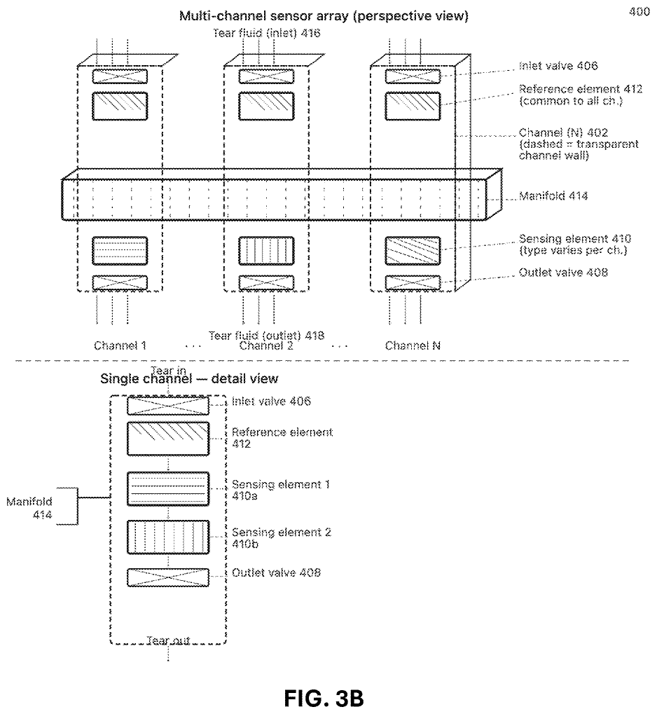

FIGS. 3A-3B illustrates the multi-channel sensor array of the i-Sense subsystem of the present disclosure. The upper portion of FIGS. 3A-3B shows a perspective view of the array comprising N parallel sensing channels, of which three representative channels are shown and designated element 402. The channels are mounted on and supported by a common manifold, element 414, which spans all channels and provides structural integrity to the array. Each channel is enclosed by transparent or semi-permeable walls, shown as dashed outlines in FIGS. 3A-3B, which permit tear fluid to enter and exit while retaining the sensing and valve elements within the channel body.

Tear fluid enters each channel from above via tear fluid inlet element 416, as indicated by the inlet flow arrows in FIGS. 3A-3B, and exits from below via tear fluid outlet element 418. The channels operate in parallel, each receiving a continuous supply of fresh tear fluid from the tear film of the subject. Element 406 depicts an inlet valve disposed at the upper opening of each channel, which regulates the flow of tear fluid into the channel and can be actuated to initiate, pause, or redirect fluid flow within the sensing array. Element 408 depicts an outlet valve disposed at the lower opening of each channel, which correspondingly regulates fluid exit and, in combination with the inlet valve, enables controlled sample presentation to the sensing elements.

Element 412 depicts a reference and calibration element disposed within each channel proximal to the inlet, which provides a reference signal against which the outputs of the analyte-specific sensing elements are normalized, compensating for variations in sample temperature, ionic strength, flow rate, and other measurement conditions. The reference element is common in design across all channels. Element 410 depicts one or more analyte-specific sensing elements disposed within each channel distal to the reference element and proximal to the outlet, each configured to continuously measure the concentration of a specific non-protein biomarker in the tear fluid passing through the channel. As shown in FIGS. 3A-3B, the sensing element type and configuration varies from channel to channel, enabling each channel to target a distinct non-protein analyte; representative differentiation between sensing element types across channels is indicated by the distinct fill patterns shown in the perspective view. In certain embodiments, each channel comprises a single sensing element; in other embodiments, each channel comprises two or more sensing elements stacked in series along the flow path, as shown in the single-channel detail view (elements 410a and 410b) in the lower portion of FIGS. 3A-3B.

The single-channel detail view shown in the lower portion of FIGS. 3A-3B illustrates the internal arrangement of one representative channel. Tear fluid enters through inlet valve (406) at the top of the channel, flows past the reference element (412), passes in sequence through sensing element 1 (410a) and sensing element 2 (410b), and exits through outlet valve (408) at the bottom of the channel. The manifold connection (414) is shown on the side of the channel. The sequential arrangement of multiple sensing elements within a single channel enables simultaneous measurement of multiple non-protein biomarkers in the same tear fluid sample passing through that channel, maximizing the information content obtained from a minimal fluid volume.

In certain embodiments, only a single valve is employed, or the pump itself serves as the valve.

### D. Integrated Circuit (IC) Chip and 3D Packaging

An integrated circuit (IC) chip is mounted on the outer surface of the posterior retention disk of the punctal plug, sized to fit within the diameter of the retention disk. The IC chip provides: (i) microwave (RF) wireless communication; (ii) control of sensors, pumps, and valves; (iii) electronic signal conditioning and data management; (iv) wireless energy harvesting; and (v) onboard power supply and regulation.

To achieve the required functionality within the constrained volume, the IC chip employs three-dimensional integrated circuit (3D IC) packaging, allowing multiple functional die—including sensor interface circuits, RF communication circuits, power management circuits, and data processing circuits—to be vertically stacked and interconnected within the limited footprint of the retention disk.

### E. Deployable Intracanalicular Antenna

In preferred embodiments, i-Sense further comprises an elongated, flexible antenna configured to extend from the punctal plug into and through the lacrimal canaliculus. The antenna is thin and flexible, designed to conform to the curvature of the canalicular lumen without causing trauma.

In a particularly preferred embodiment, the antenna is deployable: configured to slide through a lumen defined within the punctal plug, such that the plug is first inserted into the punctum and thereafter the antenna is advanced through the plug lumen into the lacrimal canaliculus. A sliding ball element is provided at the distal tip to facilitate smooth advancement and prevent trauma during deployment.

The antenna length ranges from approximately 3 mm to approximately 30 mm or greater. This extended length is of critical functional significance: wireless RF energy harvesting efficiency increases quadratically with antenna length. An antenna of 30 mm length therefore achieves up to approximately 100-fold (two orders of magnitude) greater RF energy harvesting efficiency compared to an antenna fully contained within a 3 mm plug. This quadratic efficiency scaling is an unexpected and non-obvious result of the intracanalicular deployment geometry; prior art antennas confined to punctal plug bodies do not exhibit this behavior, and no prior art reference teaches, suggests, or predicts this relationship. Applicant intends to provide experimental data confirming this unexpected result during prosecution.

### F. AcUIC Paradigm: Accuracy Under Imperfect Conditions (Overview)

A central innovation of i-Sense is the application of the Accurate Under Imperfect Conditions (AcUIC) design paradigm. Traditional biosensors are designed and calibrated assuming near-ideal test conditions—constant sample volume, stable temperature, minimal matrix interference—conditions that are not present in the continuously varying tear film environment. Sensors designed on this traditional paradigm cannot achieve clinically acceptable accuracy when deployed in the lacrimal punctum.

Under the AcUIC paradigm, all aspects of the sensing system are designed with the assumption that test conditions will be imperfect. The AcUIC-designed sensing unit incorporates the following specific measures:
(1) Microfluidic control: Pumps and valves regulate and condition the tear fluid sample prior to measurement, ensuring fresh tear delivery, appropriate sample volume, and removal of stale or contaminated fluid.
(2) Redundant sensors: Multiple sensors of each type are provided, enabling cross-validation, detection of individual sensor failure, and ensemble-based accuracy improvement.
(3) Intelligent calibration structures: Specialized nanostructures and on-chip calibration elements continuously monitor test conditions and provide reference signals for dynamic calibration.
(4) Machine learning-based correction: ML and AI algorithms analyze signals from all sensors and calibration structures jointly to compensate for identified error sources and deliver accurate results even under degraded test conditions.
(5) Error Risk Analysis (ERA): ERA is an algorithm developed by the inventor that calculates a risk factor (probability of unacceptable measurement error) for each test result. If the risk factor exceeds a predefined threshold determined by machine learning from extensive experimental data, the test result is automatically flagged and discarded, and a new measurement is initiated.

The complete AcUIC framework is described in detail below.

### G. Software and Machine Learning

The software of i-Sense implements the AcUIC framework and employs both conventional ML algorithms (neural networks, support vector machines, random forests) and Nano-Enabled AI and ML (NEAM)—a paradigm in which intelligent nanostructures physically embedded in the sensing unit provide additional physical and chemical information to ML models beyond what is available from electrical sensor signals alone. ML models are trained on historical assay data to learn compensation for variations in sample quality, environmental conditions, instrumentation errors, reagent aging, and other sources of error.

### A. AcUIC—Accurate-Test Under Imperfect Conditions

Traditional sensors are intrinsically inaccurate when placed in the tear duct or similar environments because they are designed using test protocols that assume near-perfect conditions, which are not present in field situations. The inventor has proposed and demonstrated a paradigm-shift design protocol that enables tests to be “accurate under imperfect conditions” (AcUIC). The AcUIC approach assumes that all aspects of a test by a sensor are imperfect and incorporates additional intelligent structures/devices on the sensor chip, designed specifically to gather sufficient information about test conditions. This information, combined with machine learning and other algorithms, is used to either (i) correct the imperfections and deliver an accurate test result or (ii) identify high-risk conditions that require discarding the test result and initiating a new test. Based on the AcUIC protocol, the AcUIC-based periocular sensor is not merely a sensor but a comprehensive system with the following additional features for achieving accuracy: (1) Microfluidic pumps are added to regulate and control the sample being tested (e.g., removing old tears, ensuring fresh tears, and maintaining proper tear quantity and volume). Without such precise control the sample and the test would be inaccurate. (2) Multiple sensors are added. (3) Various intelligent calibration, correction, and condition-monitoring structures are incorporated into the sensors. (4) ML, AI, and other algorithms are employed to analyze not only the signals from individual sensors but also the combined information provided by features (2) and (3) to determine an accurate result. And (5) Error Risk Analysis (ERA)—an algorithm developed by the inventor of the present invention—is implemented to calculate the risk factor (probability) in step (4). If the risk factor exceeds a predefined threshold (often determined by machine learning from extensive experiments and prior data), the test result is discarded, and a new test is initiated. The AcUIC algorithm, which employs two pathways to ensure accurate biomarker detection under imperfect conditions. The first pathway involves utilizing a multiple-sensor array rather than relying on a single sensor to measure one data point. This array provides several categories of information for analysis: (1) the primary group consists of main sensor signals used for primary biomarker detection; (2) the second group includes the sensors for temperature measurements, essential for compensating thermal variations (e.g., osmolarity); (3) the third group comprises secondary biomarkers related to the primary group; and (4) the fourth group consists of calibration structures/devices to ensure measurement consistency. Additionally, supporting sensors monitor the measurement environment—such as voltage, current, and related physical or biomarker parameters—to ensure optimal operating conditions.

Furthermore, in AcUIC, even the primary sensing data is not derived from a single sensor but from an array of redundant sensors of similar types with varying sizes and operating conditions. This redundancy enhances reliability and enables comparative analysis. Measurements are taken multiple times, not just once, and are controlled by a pump. The pump can flush out the tear fluid to clear the channel, allowing the sensor to compare signals with and without the sample. This comparison is essential for signal validation and accuracy.

All sensor and environmental data are stored in a reference library, which helps determine—using an AI-powered model—how to correct the test data and whether the measurement was conducted under acceptable conditions. After collecting and analyzing the data, an AI-based signal analysis module produces the initial measurement result.

The second pathway in AcUIC involves a second AI module, which evaluates the trustworthiness of that result by calculating the probability that the data might be incorrect. If this probability exceeds a preset threshold, the result is discarded, and the measurement is repeated until the confidence level is satisfactory.

Once a trustworthy result is obtained, it is sent to i-TheraX for therapeutic decision-making, such as determining drug dosage. This algorithm enables accurate measurement even under imperfect conditions. Furthermore, a training database is built using gold-standard measurements to establish how specific parameters behave under various conditions.

The system can also incorporate external data sources, such as smartwatches, which provide physiological information (e.g., fever, hydration) and environmental conditions that may affect tear biomarker levels. This further improves measurement accuracy and reliability. For example, elevated body temperature could impact osmolality readings relevant to dry eye diagnostics.

An example of the devices used in AcUIC includes a primary sensor for osmolarity, with several such sensors with different dimension arranged within a channel. The second group comprises temperature sensors. The third group consists of sensors for ion concentrations (e.g., Na⁺, Cl⁻, K⁺, Ca²⁺), pH levels, or glucose that have connections to osmolarity. The fourth group is the monitors of voltage, current, device geometry, and sampling conditions. Multiple sample characteristics will be monitored in parallel to verify and maintain proper testing conditions.

### i-Release: Closed-Loop Drug Delivery Subsystem

### A. Structural Overview

i-Release is an intelligent, accurate, closed-loop drug delivery device shaped as a plug configured for implantation within the lacrimal punctum, either in the same punctum as i-Sense or in a second punctum of the same or contralateral eye. i-Release comprises hardware and software for controlled release of therapeutic agents based on sensing information received from i-Sense and dosing instructions from i-TheraX.

### B. Drug Storage and Delivery Mechanisms

The i-Release plug defines an internal chamber configured to store therapeutic agent in solid or liquid form:
(a) Liquid drug storage: The chamber is filled with a liquid drug formulation. A microfluidic pump delivers the liquid drug into the periocular tissue at a controlled rate and volume.
(b) Solid drug storage: A portion of the chamber is filled with a solid (e.g., lyophilized or crystalline) drug formulation. A separate mixing sub-chamber is also defined within the plug. Upon a dosing instruction, the pump draws tear fluid from the eye into the mixing sub-chamber to dissolve a controlled quantity of the solid drug, which is then pumped into the periocular region. This approach extends operational lifetime between refills or replacements.
(c) Hybrid storage: Combinations of solid and liquid drug storage are also within the scope of the invention.

Active inlet and outlet valves prevent passive diffusion of drug in the absence of a dosing instruction, providing precise dose control and preventing inadvertent overdosing.

### C. Acoustic Pump and Alternatives

In preferred embodiments, i-Release employs an acoustic pump —an acoustically driven microfluidic pump using ultrasonic vibrations to propel fluid —for controlled drug mixing and delivery. In alternative embodiments, piezoelectric pumps, electroosmotic pumps, or combinations thereof may be used.

### D. Therapeutic Agents

i-Release is capable of delivering a wide range of therapeutic agents to the periocular region. In preferred embodiments for dry eye disease applications, initial therapeutic agents include cyclosporine (an immunosuppressive agent reducing ocular surface immune reactions and inflammation) and lacritin (a tear protein with tear film stabilizing and anti-inflammatory properties).

### i-TheraX: Therapeutic Algorithm Subsystem

i-TheraX is a software algorithm subsystem that determines dosing instructions—specifying dose amount and timing—transmitted to i-Release. i-TheraX bases its dosing instructions on integration of: (i) real-time biomarker sensing information from i-Sense; (ii) contextual health and behavioral information from paired smartwatch and smartphone sensors (e.g., physical activity, sleep patterns); (iii) patient-specific and population-level treatment databases; and (iv) AI-trained therapeutic models developed through clinical studies and animal model validation.

i-TheraX implements a closed-loop control architecture in which the biomarker response to a delivered dose is used to update the therapeutic model, enabling personalized, adaptive dosing that improves over time for each individual patient.

FIGS. 4A-4B illustrates the extendable antenna of the i-NanoOcuCare system of the present disclosure, shown in anatomical context within the lacrimal canaliculus. Element 518 depicts the lacrimal punctum, the natural opening in the eyelid margin through which the plug is implanted. Element 504 depicts the retention disk of the punctal plug, which sits approximately flush with, or slightly proud of, the eyelid surface at the punctum. Element 506 depicts the plug body, which is retained within the vertical portion of the lacrimal canaliculus (element 514). Element 508 depicts the sensing chip disposed on the plug body within the vertical canaliculus, as described in connection with FIG. 2 and FIGS. 3A-3B. The lacrimal canaliculus curves from its vertical portion (514) at the punctum to a horizontal portion (element 516), which extends medially within the eyelid tissue toward the lacrimal sac. The transition from vertical to horizontal canaliculus follows the natural anatomy of the lacrimal drainage system.

Element 520 depicts the antenna connection point, located at the anterior end of the plug body. Element 510 depicts the flexible extendable antenna, a thin, flexible elongated conductor that exits the plug at the antenna connection point, follows the curve of the canaliculus from the vertical to the horizontal portion, and extends along the horizontal canaliculus. The antenna is flexible to conform to the natural curvature of the lacrimal canaliculus without causing discomfort or tissue damage. In certain embodiments, the antenna comprises two parallel conductors, one for wireless signal transmission and one for wireless energy harvesting, as indicated by the parallel lines shown in FIGS. 4A-4B. The antenna may further comprise periodic flexible joint segments along its length to facilitate routing through the canaliculus, as indicated by the segment markers shown in FIGS. 4A-4B.

Element 512 depicts a guiding ball disposed at the distal tip of the antenna. The guiding ball has a smooth, rounded surface and a diameter slightly smaller than the internal diameter of the horizontal canaliculus, such that it guides the antenna tip smoothly through the canaliculus during insertion without catching on the canalicular walls or tissue folds.

As indicated by the dimension annotations in FIGS. 4A-4B, the plug body has a length of approximately 3 mm within the canaliculus. The extendable antenna may be extended to a length of up to approximately 30 mm—up to ten times the internal plug length—through the horizontal canaliculus. Because the power that can be harvested by a receiving antenna scales with the square of the antenna length, a ten-fold increase in antenna length corresponds to an increase in harvested power of up to approximately 100-fold relative to an antenna confined within the plug body alone, as noted in the energy annotation in v. These values are illustrative; the achievable energy gain depends on the antenna geometry, tissue coupling, and the frequency and field strength of the external power source.

As shown in the two configuration insets at the lower portion of FIGS. 4A-4B, the extendable antenna may be implemented in either of two configurations. In configuration (a), the fixed antenna configuration, the antenna is permanently attached to the plug prior to implantation and is deployed into the horizontal canaliculus together with the plug in a single insertion procedure. In configuration (b), the insertable antenna configuration, the plug is first positioned within the vertical canaliculus, and the antenna is subsequently inserted through a longitudinal channel within the plug body and advanced into the horizontal canaliculus as a separate step. The insertable configuration allows the antenna length to be adjusted to the individual anatomy of the subject's canaliculus, and permits antenna replacement or repositioning without removing the plug. Both configurations are encompassed by the present disclosure.

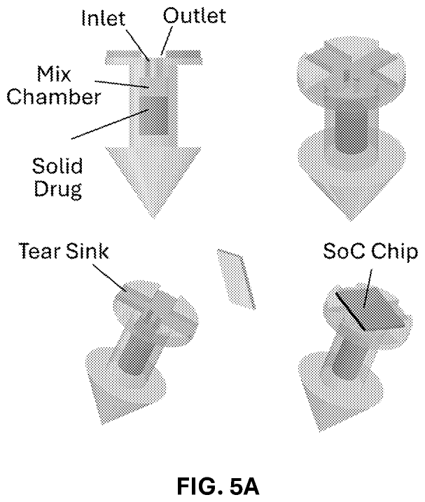

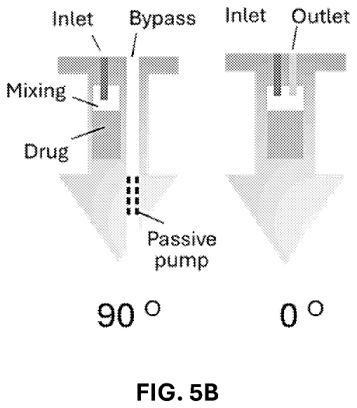

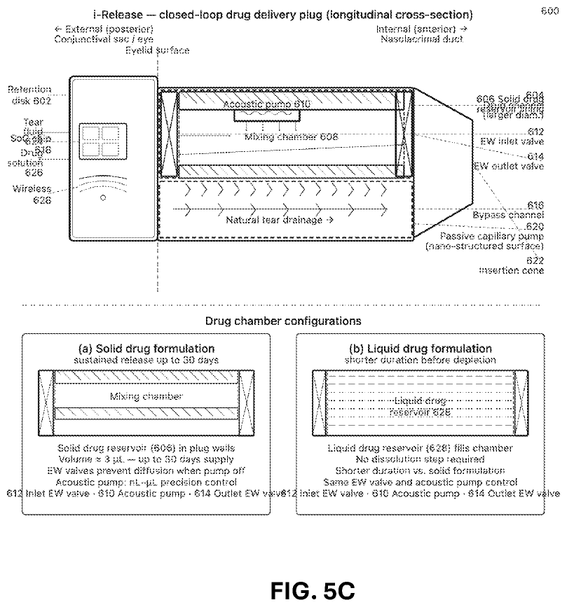

FIGS. 5A-5C illustrate the i-Release closed-loop drug delivery plug of the present disclosure. The i-Release plug has the same overall form factor as the i-Sense plug described in connection with FIG. 2, comprising a posterior retention disk, a central tubular body, and a front insertion cone configured for implantation in the lacrimal punctum. The drug delivery architecture replaces the sensing channel of the i-Sense plug with a larger-diameter drug channel, as described below.

Element 602 depicts the posterior retention disk, which sits approximately flush with, or slightly proud of, the eyelid surface when the plug is implanted. Element 618 depicts the SoC chip integrated within the retention disk, which provides onboard computation, control of the electrowetting valves and acoustic pump, and wireless communication with the external i-TheraX system. Element 628 depicts the wireless communication element on the face of the retention disk, which enables the i-Release plug to receive AI-generated dosing instructions from i-TheraX and to transmit operational status data.

Element 604 depicts the drug channel, which occupies the larger-diameter upper portion of the plug body and replaces the sensing channel of the i-Sense plug. The drug channel comprises, from the inlet to the outlet, the following elements in series. Element 612 depicts the electrowetting (EW) inlet valve, disposed at the inlet of the drug channel, which regulates the entry of tear fluid into the drug channel and, when closed, prevents diffusion of dissolved drug from the mixing chamber to the exterior of the plug. Element 606 depicts the solid drug reservoir, which lines the inner walls of the drug channel in the form of a compacted solid drug layer embedded within the inner diameter of the plug body. Tear fluid pumped into the drug channel by the acoustic pump contacts the solid drug reservoir and dissolves a controlled quantity of drug. Element 608 depicts the mixing chamber, the open central space within the drug channel between the solid drug reservoir linings, in which tear fluid and dissolved drug are mixed by the acoustic pump. Element 610 depicts the acoustic pump, a transducer disposed on the wall of the drug channel that generates acoustic waves directed into the mixing chamber. The acoustic pump provides bidirectional, precision flow control from nanoliters to microliters, enabling metered delivery of dissolved drug solution. In addition to its pumping function, the acoustic wave field provides an anti-fouling mechanism that prevents biofouling of the drug channel surfaces during extended operation. Element 614 depicts the electrowetting (EW) outlet valve, disposed at the outlet of the drug channel, which regulates the release of drug solution from the mixing chamber to the exterior of the plug and, when closed, isolates the mixing chamber to prevent unintended diffusion of dissolved drug. The electrowetting valves are designed to operate at very low power, in certain embodiments as few as several microwatts, enabling sustained operation within the wireless power budget of the implanted plug.

As indicated by the flow arrows in FIGS. 5A-5C, tear fluid (element 624) enters the drug channel through the EW inlet valve (612) and is drawn into the mixing chamber (608) by the acoustic pump (610), where it dissolves drug from the solid reservoir lining (606). The resulting drug solution is released through the EW outlet valve (614) back toward the conjunctival sac as drug solution (element 626), delivering the therapeutic agent directly to the ocular surface. The drug channel operation is controlled by AI-generated dosing instructions received wirelessly from i-TheraX, specifying the dose amount, timing, and duration of each delivery cycle.

Element 616 depicts the bypass channel, which occupies the smaller-diameter lower portion of the plug body and runs parallel to the drug channel along the length of the plug. The bypass channel provides a continuous pathway for natural tear drainage through the plug from the conjunctival sac to the nasolacrimal duct, compensating for the reduction in effective canalicular cross-section caused by the drug channel. Element 620 depicts a passive capillary pump disposed within the bypass channel, comprising micro-and nanostructured surfaces that drive tear fluid flow by capillary action without requiring any moving parts or external power.

The lower portion of FIGS. 5A-5C shows the two drug chamber configurations of the i-Release system. Configuration (a) shows the solid drug formulation, in which a solid drug reservoir lines the walls of the drug channel, providing a drug supply that can sustain therapeutic delivery for up to approximately 30 days within the approximately 3 μL volume of the plug. Configuration (b) shows the liquid drug formulation, in which a liquid drug reservoir fills the drug chamber directly, without a dissolution step. The liquid drug configuration is simpler in operation but provides a shorter duration of drug supply before depletion. Both configurations employ the same electrowetting valve and acoustic pump control architecture. In both cases, the system volume is approximately 3 μL, consistent with the millimeter-scale dimensions of the plug body.

### Wireless Communication and Power

Wireless communication and power management functions are distributed between the onboard IC chip(s) and a pocket-sized wireless station external to the eye. The wireless architecture comprises two antenna links (downlink and uplink) operating at 2.4 GHz (ISM band) in preferred embodiments, with a downlink energy efficiency target of less than 1 pJ/bit. The architecture provides: (1) wireless power delivery via RF energy harvesting; (2) bidirectional wireless data communication between the plug and the external station; (3) power management including buffering of instantaneous peak current demands; (4) signal acquisition and conditioning; and (5) data security using encryption with integrity verification.

Power may also be supplemented by Peltier thermoelectric generation (exploiting the temperature differential between the ocular surface and ambient air) and/or photovoltaic conversion.

### Pocket-Sized Wireless Station

A pocket-sized wireless station (approximately smartphone-sized) communicates wirelessly with the punctum-embedded device(s) and provides imaging, communication, and computation (ICCOM) functions. The station communicates bidirectionally with: the punctum-embedded sensing and drug delivery plugs; cloud computing infrastructure (e.g., Amazon Web Services) via HTTPS with TLS 1.2/1.3 encryption; and the patient's smartwatch and smartphone. Medical data is transmitted in HL7 FHIR format for interoperability with electronic health records. The station performs front-end ML inference, data management, and signal processing to reduce the computational burden on the constrained onboard IC chip.

### AI, Machine Learning, and Nano-Enabled AI and ML (NEAM)

The i-NanoOcuCare platform employs ML and AI throughout all subsystems. The ML approaches include both conventional ML algorithms and Nano-Enabled AI and ML (NEAM)—a paradigm in which intelligent nanostructures physically embedded in the sensing and delivery chips provide additional physical and chemical information to ML models beyond what is available from electrical signals alone. NEAM enables ML models to leverage nanoscale physical phenomena for superior pattern recognition and error correction.

Key ML functions within the platform include: correction of sensor bias and drift; identification of anomalous or unreliable measurements; real-time dynamic calibration; integration of tear biomarker data with wearable sensor data for systemic disease assessment; personalization of therapeutic dosing models; and continual model updating as patient data accumulates.

### Dry Eye Disease Application and Animal Model Validation

In a primary embodiment, the i-NanoOcuCare platform is applied to the continuous monitoring and closed-loop treatment of dry eye disease (DED). Tear osmolarity—a key biomarker of DED—is monitored as the primary sensing parameter, supplemented by inflammatory protein biomarkers and glucose. The i-TheraX algorithm determines cyclosporine or lacritin dosing based on measured biomarker profiles. Animal model studies are used to validate the i-TheraX dosing algorithm and to establish treatment response models prior to human clinical trials. The platform is not limited to DED; other ocular conditions amenable to tear biomarker monitoring and periocular drug delivery are also within the scope.

### Relationship to Prior iMOST Platform

The i-NanoOcuCare platform is an extension of and successor to the iMOST (integrated Microfluidic Ocular Sensing and Treatment) platform previously developed by the inventor, as illustrated in FIGS. 3A-3B. The iMOST platform established foundational microfluidic and sensing architectures for punctum-embedded ocular biosensing. The present invention substantially advances iMOST by adding: the full AcUIC accuracy framework with ERA; closed-loop drug delivery via i-Release; AI-driven therapeutics via i-TheraX; a deployable intracanalicular antenna; and a complete wireless system architecture including cloud and wearable connectivity.

### AcUIC Framework: Detailed Description

This section provides a complete, standalone detailed description of the Accurate Under Imperfect Conditions (AcUIC) sensing methodology developed by the inventor. The AcUIC methodology is both (a) an integral subsystem of the i-NanoOcuCare platform as described in Sections I through IX above, and (b) a standalone invention applicable to any periocular or other biological fluid sensing context in which operating conditions are inherently imperfect. The standalone claims for the AcUIC methodology are reserved in Section XVI for a planned continuation or divisional application.

### A. Conceptual Foundation

The AcUIC framework is founded on the recognition that accurate periocular biomarker assay in a real-world, in vivo environment cannot be achieved by sensor designs that assume near-perfect operating conditions. The AcUIC framework deliberately assumes that all aspects of the test—including sample composition, sample volume, temperature, electrode state, and device geometry—are subject to imperfection and variability, and incorporates the detection, quantification, and correction of these imperfections as first-class design elements of the sensor system.

The AcUIC approach proceeds through the following operational stages: (1) sample preparation and microfluidic control; (2) multi-sensor acquisition; (3) condition monitoring; (4) Error Risk Analysis; and (5) result delivery or test cycle restart. Each stage is described below.

### B. Microfluidic Control Subsystem

A critical source of inaccuracy in periocular sensing is uncontrolled sample state—including stale tear fluid, variable tear volume, and contamination from mucus or cellular debris. The AcUIC sensor platform incorporates one or more on-chip microfluidic pumps configured to: (a) flush and remove old or stagnant tear fluid from the sensing region prior to each test cycle; (b) ensure delivery of fresh, representative tear fluid to the sensing elements; and (c) regulate and maintain a defined, controlled tear volume at the sensor surface during the measurement window. In exemplary embodiments, the microfluidic pump may be implemented as an electroosmotic pump, a piezoelectric micropump, a thermopneumatic pump, or any other suitable microfluidic actuation mechanism integrated onto the sensor chip. The microfluidic control subsystem is operatively coupled to the condition-monitoring elements and the ERA module so that pump operation itself can be monitored and accounted for in the risk analysis.

### C. Multiple Sensing Elements and Redundancy

To overcome the limitations of single-sensor designs—which are inherently susceptible to individual electrode drift, fouling, or failure—the AcUIC sensor platform incorporates a plurality of sensing elements. In one embodiment, the plurality of sensors includes multiple electrochemical electrodes independently measuring one or more of: osmolarity, Na⁺ concentration, K⁺ concentration, Cl⁻ concentration, Ca²⁺ concentration, pH, and local sample temperature.

Multiple sensors serve two complementary functions. First, redundancy: if one sensor is determined by the ERA module to be operating outside its reliable range, remaining sensors may still provide a sufficient basis for an accurate result. Second, cross-sensor signal correlations provide a rich information source for the condition-monitoring and correction algorithms, enabling detection of systematic errors as distinct from random noise.

### D. Calibration, Correction, and Condition-Monitoring Structures

The AcUIC sensor platform integrates directly onto the sensor chip a suite of intelligent calibration, correction, and condition-monitoring structures, including:
Reference electrodes and on-chip calibration standards for real-time electrode drift correction;
Temperature sensors for local thermal monitoring and temperature-dependent signal correction;
Impedance spectroscopy elements for monitoring electrode-electrolyte interface condition, including fouling and surface degradation;
Applied voltage and current monitoring elements to detect deviations from expected electrochemical behavior;
Optical or capacitive volume sensors for confirming adequate sample presence; and
Device geometry and structural integrity monitors to detect displacement, mechanical deformation, or improper seating of the sensor at the punctum.

Together, these structures generate a comprehensive, real-time description of test conditions, which serves as the primary input to the ERA module and the correction algorithms.

### E. Machine Learning and Signal Processing Pipeline

The AcUIC framework employs machine learning (ML), artificial intelligence (AI), and other algorithms to process the multi-channel data stream generated by the multiple sensing elements and condition-monitoring structures. The signal processing pipeline comprises:
(a) Feature Extraction: Relevant features are extracted from the raw multi-sensor data stream, including signal magnitudes, time derivatives, cross-sensor correlations, impedance spectra characteristics, and condition-monitoring parameter values.
(b) Signal Correction Model: A trained ML model—such as a neural network, random forest, gradient boosted ensemble, or Gaussian process regressor—maps the raw sensor signals and condition features to a corrected analyte concentration estimate that compensates for known sources of imperfection including electrode drift, temperature variation, matrix effects, and fouling.
(c) Uncertainty Quantification: The ML model also outputs an uncertainty estimate associated with the corrected result, which contributes to the ERA risk factor calculation.
In preferred embodiments, the ML models are trained using a large dataset of validated measurements obtained under a wide range of controlled and purposely imperfect conditions, using validated reference standard measurements as ground truth labels.

### F. Error Risk Analysis (ERA)

A central and novel feature of the AcUIC invention is the Error Risk Analysis (ERA) module. ERA provides a systematic, quantitative framework for computing the probability—the risk factor—that a given test result falls outside an acceptable accuracy threshold.

In one embodiment, the ERA module computes a scalar risk factor R∈[0, 1] as a function of: (i) the condition-monitoring parameters (e.g., temperature deviation, electrode impedance anomaly, volume insufficiency indicator); (ii) the uncertainty estimate from the ML correction model; and (iii) historical statistical data characterizing the relationship between observed condition parameters and actual measurement accuracy, derived from the training dataset.

The risk factor R is compared to a predefined decision threshold T, which is itself determined from the training data using ML optimization. If R≤T, the corrected assay result is accepted and reported. If R>T, the assay result is flagged as unreliable, discarded, and a new test cycle is automatically initiated after the microfluidic control subsystem has re-prepared the sample.

This dual-outcome decision structure—accurate result delivery or controlled test restart—is a key innovation of the AcUIC framework and distinguishes it from prior art that either reports unreliable results uncritically or simply flags errors without providing a corrective action pathway.

### G. Exemplary Embodiment: Punctal Plug Osmolarity and Ion Sensing

In a specific and exemplary embodiment of the standalone AcUIC system, the sensor is implemented as a punctal plug device configured for chronic, minimally invasive deployment in the lacrimal punctum. The punctal plug sensor detects osmolarity as the primary clinical analyte, and additionally detects any one or more of: sample temperature, Na⁺, Cl⁻, K⁺, Ca²⁺ concentrations, pH, applied voltage, applied current, and device condition parameters. Osmolarity is measured via impedance-based conductivity sensing; individual ion concentrations are measured using ion-selective electrodes; pH via a solid-state pH-sensitive electrode.

### H. Training and Validation Protocol

The ML models and ERA threshold parameters are established through a structured multi-stage experimental protocol: (a) controlled experiments exposing the AcUIC sensor to tear fluid analogs of known composition under purposely varied imperfect conditions with validated reference measurements; (b) training the correction model and establishing the risk threshold T by cross-validation; and (c) validation in independent experiments and, ultimately, in clinical studies with human subjects. The training dataset is contemplated to comprise at least hundreds, and preferably thousands, of experimental measurements spanning the anticipated range of real-world imperfect conditions.

### I. Broader Applicability of AcUIC

While the detailed description of AcUIC primarily addresses tear fluid and the punctal plug embodiment, the AcUIC framework is broadly applicable to any biological fluid sensing scenario in which operating conditions are subject to real-world imperfection, including saliva-based sensors, wound exudate sensors, transdermal sensors, and similar modalities.

While the foregoing written description of the invention enables one of ordinary skill to make and use what is considered to be the best modes thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

What is claimed is:

1. A punctum-embedded ocular sensing device, comprising:
a punctal plug configured to be implanted within a lacrimal punctum of an eye, the punctal plug having a proximal end configured for insertion into the lacrimal punctum and a distal end comprising a retention disk;
a first channel defined within the punctal plug and configured to direct tear fluid through the punctal plug for biomarker sensing;
a sensing unit disposed within the first channel, the sensing unit comprising a nano sensor array configured to detect at least one biomarker in the tear fluid;
a microfluidic pump and an active fluidic valve operatively coupled to the sensing unit and configured to control a flow of tear fluid into, through, and out of the sensing unit;
an integrated circuit chip mounted on the retention disk, the integrated circuit chip configured to provide wireless communication, electronic signal control, wireless energy harvesting, and onboard power supply; and
memory storing instructions that, when executed by the integrated circuit chip, cause the integrated circuit chip to execute machine learning algorithms configured to process signals from the nano sensor array and produce biomarker measurements that are accurate under imperfect tear sampling conditions.

2. The punctum-embedded ocular sensing device of claim 1, further comprising a second channel defined within the punctal plug and configured as a bypass channel to allow tear fluid to flow from a conjunctival sac to a nasolacrimal duct without passing through the sensing unit.

3. The punctum-embedded ocular sensing device of claim 2, wherein the second channel further comprises a passive microfluidic flow accelerator configured to increase tear flow rate through the bypass channel without requiring external power.

4. The punctum-embedded ocular sensing device of claim 1, wherein the integrated circuit chip employs three-dimensional integrated circuit (3D IC) packaging to accommodate a plurality of functional circuit elements within a footprint substantially fitting within a diameter of the retention disk.

5. The punctum-embedded ocular sensing device of claim 1, further comprising a deployable intracanalicular antenna comprising an elongated flexible antenna element configured to be advanced through a lumen of the punctal plug into a lacrimal canaliculus, wherein the elongated flexible antenna element has a length between approximately 3 mm and approximately 30 mm or greater.

6. The punctum-embedded ocular sensing device of claim 5, further comprising a sliding ball element at a distal tip of the elongated flexible antenna element, wherein the sliding ball element is configured to facilitate smooth deployment of the deployable intracanalicular antenna through the lacrimal canaliculus.

7. The punctum-embedded ocular sensing device of claim 1, wherein the nano sensor array is configured to detect at least two biomarkers selected from the group consisting of: osmolarity, glucose, lactate, urea, sodium ion, potassium ion, calcium ion, chloride ion, and a tear protein biomarker.

8. The punctum-embedded ocular sensing device of claim 1, wherein the nano sensor array comprises at least one enzyme-based electrochemical sensor (EAEC), at least one ion-selective electrode (ISE) sensor, and at least one immunoassay sensor.

9. The punctum-embedded ocular sensing device of claim 8, wherein the at least one immunoassay sensor is configured for continuous immunoassay measurement by applying an AC electric field to periodically dissociate bound analyte-antibody complexes and regenerate a surface of the at least one immunoassay sensor.

10. The punctum-embedded ocular sensing device of claim 1, wherein the machine learning algorithms implement an Error Risk Analysis (ERA) method configured to:
calculate a risk factor representing a probability that a given measurement result exceeds a predefined accuracy error threshold; and
automatically discard the given measurement result and initiate a new measurement when the risk factor exceeds the predefined accuracy error threshold.

11. The punctum-embedded ocular sensing device of claim 1, wherein the machine learning algorithms implement Nano-Enabled AI and ML (NEAM), wherein intelligent nanostructures disposed within the sensing unit provide physical and chemical condition information to the machine learning algorithms in addition to electrical sensor signals.

12. The punctum-embedded ocular sensing device of claim 1, wherein the sensing unit further comprises a plurality of parallel sensing sub-channels, and wherein the microfluidic pump and active fluidic valve are configured to open only a single sub-channel for sensing at a time while keeping remaining sub-channels closed, thereby reducing reagent degradation and sensor fouling.

13. A closed-loop ocular care system, comprising:
a sensing plug configured for implantation within a lacrimal punctum and comprising a nano sensor array configured to continuously detect a plurality of biomarkers in tear fluid, a microfluidic pump, an active fluidic valve, and an integrated circuit chip;
a drug delivery plug configured for implantation within a lacrimal punctum and comprising a drug storage chamber, a drug delivery pump, at least one active valve, and an integrated circuit chip;
a therapeutic algorithm subsystem configured to receive biomarker data from the sensing plug, determine dosing instructions based on said biomarker data and a therapeutic model, and transmit said dosing instructions to the drug delivery plug; and
a wireless communication station configured for bidirectional wireless communication with both the sensing plug and the drug delivery plug, and further configured for communication with a cloud computing platform and at least one wearable device;
wherein the sensing plug, the drug delivery plug, the therapeutic algorithm subsystem, and the wireless communication station operate in concert to continuously monitor ocular biomarker status and automatically deliver therapeutic agents in a closed loop.

14. The closed-loop ocular care system of claim 13, wherein the drug delivery plug is configured to store a solid therapeutic agent, and wherein the drug delivery pump is configured to draw tear fluid into a mixing chamber of the drug delivery plug to dissolve a controlled quantity of the solid therapeutic agent prior to delivery.

15. The closed-loop ocular care system of claim 13, wherein the drug delivery pump is an acoustic pump driven by ultrasonic vibrations.

16. The closed-loop ocular care system of claim 13, wherein the therapeutic algorithm subsystem is further configured to receive contextual health data from at least one wearable device and to incorporate said contextual health data into the dosing instructions.

17. The closed-loop ocular care system of claim 13, wherein the wireless communication station is configured to transmit medical data in HL7 FHIR format to a cloud computing platform via an encrypted HTTPS connection.

18. The closed-loop ocular care system of claim 13, wherein the integrated circuit chip of the sensing plug is configured to harvest wireless RF energy from a deployable intracanalicular antenna having a length between approximately 3 mm and approximately 30 mm, wherein energy harvesting efficiency increases quadratically with antenna length.

19. A method for closed-loop ocular monitoring and treatment, comprising:
implanting a sensing plug within a lacrimal punctum of an eye of a patient, the sensing plug comprising a nano sensor array;
continuously drawing tear fluid through a sensing channel of the sensing plug using a microfluidic pump;
detecting one or more signals corresponding to a plurality of biomarkers in the tear fluid using the nano sensor array;
processing the one or more signals using a machine learning algorithm executing on an integrated circuit chip of the sensing plug to produce biomarker measurements compensated for imperfect test conditions;
transmitting the biomarker measurements to a therapeutic algorithm;
generating dosing instructions from the therapeutic algorithm based on the biomarker measurements; and
delivering a therapeutic agent to a periocular region of the eye of the patient via a drug delivery plug implanted in a lacrimal punctum, in an amount and at a time specified by the dosing instructions.

20. The method of claim 19, further comprising:
calculating, using an Error Risk Analysis algorithm, a risk factor for each of the biomarker measurements, and discarding those biomarker measurements for which the risk factor exceeds a predefined threshold; and
repeating the detecting and processing steps when the risk factor exceeds a predefined threshold.

21. The method of claim 19, further comprising:
receiving health and behavioral data from a wearable device worn by the patient; and
incorporating the health and behavioral data into the generating of dosing instructions.

22. The method of claim 19, wherein the plurality of biomarkers comprises at least osmolarity and at least one of glucose, lactate, urea, an electrolyte ion, or a protein biomarker.

23. A deployable intracanalicular antenna system for a punctum-embedded medical device, comprising:
a punctal plug configured to be implanted within a lacrimal punctum, the punctal plug having a punctal plug body defining an internal lumen extending longitudinally therethrough;
an elongated flexible antenna element configured to be slidably advanced through the internal lumen of the punctal plug and into a lacrimal canaliculus after implantation of the punctal plug, wherein the antenna element has a length greater than the length of the punctal plug; and
a distal tip element attached to a distal end of the elongated flexible antenna element, the distal tip element configured to facilitate smooth advancement of the elongated flexible antenna element through the lacrimal canaliculus;
wherein wireless energy harvesting efficiency of the antenna element increases quadratically with antenna length, enabling wireless RF power harvesting at an efficiency at least 10-fold greater than achievable by an antenna confined to the punctal plug body alone.

24. The deployable intracanalicular antenna system of claim 23, wherein the elongated flexible antenna element has a length between approximately 10 mm and approximately 30 mm, achieving wireless energy harvesting efficiency at least 10-fold greater than an antenna confined to the punctal plug body.

25. The deployable intracanalicular antenna system of claim 23, wherein the distal tip element is a spherical or rounded ball configured to reduce friction with epithelial lining of the lacrimal canaliculus during deployment.
